(19) United States
(12) Patent Application Publication
(10) Pub. No.: US 2019/0358838 A1
(43) Pub. Date: Nov. 28, 2019

(54) BLADE GRIP ASSEMBLY

(71) Applicant: AccuTec Blades, Inc., Verona, VA (US)

(72) Inventor: Jeffrey W. WONDERLEY, Fort Defiance, VA (US)

(21) Appl. No.: 16/484,396

(22) PCT Filed: Feb. 7, 2019
(86) PCT No.: PCT/US2018/017298

§ 371 Date: Aug. 7, 2019

Related U.S. Application Data
(60) Provisional application No. 62/456,061, filed on Feb. 7, 2017.

(51) Int. Cl. B26B 21/56; A61B 17/3205; B26B 21/08; B26B 21/40

(52) U.S. Cl. CPC B26B 21/565; A61B 17/3205; A61B 10/0233; B26B 21/40; B26B 21/08

(57) ABSTRACT

A blade grip assembly includes a blade having at least one cutting edge and at least one blade grip coupled to the blade. The blade grip includes a body and a plurality of teeth extending from the body. The plurality of teeth is on a plane different from a plane of the blade when the blade is in an unflexed position.

### BACKGROUND

The present disclosure relates generally to a blade grip assembly for a razor blade.

Certain services, such as dermatological services, rely on a flexibility of razor blades to perform certain procedures. For example, a dermatologist may grip blunt sides of a razor blade between a thumb and an additional gripping finger to flex the razor blade in a manner that limits a cutting surface of the razor blade to provide cutting accuracy. Flexing the razor blade by moving the thumb and the additional gripping finger toward each other results in less of the razor blade being placed in contact with an object being cut. However, such gripping of the razor blade may increase a likelihood of injuries to the dermatologist or other user that grips and flexes the razor blade. Further, gripping the razor blade in this way may result in the blade slipping from the grip of the user, thereby causing less accurate cuts.

### SUMMARY

The disclosed embodiments provide blade grips and assemblies that include the blade grips. In accordance with an embodiment, a symmetrical blade grip assembly includes a razor blade and two blade grips coupled symmetrically to the razor blade. The two blade grips are coupled to the razor blade at opposite ends of the razor blade.

In accordance with another illustrative embodiment, an asymmetrical blade grip assembly includes a razor blade and two blade grips coupled asymmetrically to the razor blade. The two blade grips are coupled to the razor blade at opposite ends of the razor blade.

In another illustrative embodiment, a blade grip assembly includes a blade having at to least one cutting edge and a pair of blade grips coupled to opposing edges of the blade. Each blade grip includes a body and a plurality of teeth extending from the body. The plurality of teeth is on a plane different from a plane of the blade when the blade is in an unflexed position.

In yet another illustrative embodiment, a blade grip assembly includes a blade having at least one cutting edge and a pair of blade grips coupled to opposing edges of the blade. Each of the blade grips includes a body and a plurality of teeth extending from the body. The plurality of teeth is on a plane angled relative to a plane of the blade when the blade is in an unflexed position.

Additional details of the disclosed embodiments are provided below in the detailed description and corresponding drawings.

### BRIEF DESCRIPTION OF THE DRAWINGS

Illustrative embodiments of the present invention are described in detail below with reference to the attached figures, which are incorporated by reference herein, and wherein:

FIG. 1 is a perspective view of a razor blade in a flexed position, in accordance with an embodiment;

FIG. 2 is a top perspective view of a symmetrical blade grip assembly including the razor blade of FIG. 1 shown in an unflexed position, in accordance with an embodiment;

FIG. 3 is a bottom perspective view of an underside of the symmetrical blade grip assembly of FIG. 2, in accordance with an embodiment;

FIG. 4 is a front view of the symmetrical blade grip assembly of FIG. 2, in accordance with an embodiment;

FIG. 5 is a side view of a grip of the symmetrical blade grip assembly of FIG. 2, in accordance with an embodiment;

FIG. 6 is a top perspective view of an asymmetrical blade grip assembly including the razor blade of FIG. 1 shown in an unflexed position, in accordance with an embodiment;

FIG. 7 is a bottom perspective view of an underside of the asymmetrical blade grip assembly of FIG. 6, in accordance with an embodiment;

FIG. 8 is a front view of the asymmetrical blade grip assembly of FIG. 6, in accordance with an embodiment;

FIG. 9 is a left side view of a grip of the asymmetrical blade grip assembly of FIG. 6, in accordance with an embodiment; and

FIG. 10 is a flow chart of a method of manufacturing the blade grip assemblies of FIGS. 2 and 6, in accordance with an embodiment.

The illustrated figures are only exemplary and are not intended to assert or imply any limitation with regard to the environment, architecture, design, or process in which different embodiments may be implemented.

### DETAILED DESCRIPTION

In the following detailed description of several illustrative embodiments, reference is made to the accompanying drawings that form a part hereof, and in which is shown by way of illustration specific embodiments in which the invention may be practiced. These embodiments are described in sufficient detail to enable those skilled in the art to practice the invention, and it is understood that other embodiments may be utilized and that logical structural, mechanical, electrical, and chemical changes may be made without departing from the spirit or scope of the invention. To avoid detail not necessary to enable those skilled in the art to practice the embodiments described herein, the description may omit certain information known to those skilled in the art. The following detailed description is, therefore, not to be taken in a limiting sense, and the scope of the illustrative embodiments are defined only by the appended claims.

Unless otherwise specified, any use of any form of the terms “connect,” “engage,” “couple,” “attach,” or any other term describing an interaction between elements is not meant to limit the interaction to direct interaction between the elements and may also include indirect interaction between the elements described. Further, any use of any form of the terms “connect,” “engage,” “couple,” “attach,” or any other term describing an interaction between elements includes items integrally formed together without the aid of extraneous fasteners or joining devices. In the following discussion and in the claims, the terms “including” and “comprising” are used in an open-ended fashion, and thus should be interpreted to mean “including, but not limited to”. Unless otherwise indicated, as used throughout this document, “or” does not require mutual exclusivity.

The subject matter disclosed in the present application provides an assembly for securely grasping a razor blade. In specific applications, it is desirable for a user of a razor blade to securely grasp non-cutting edges of a razor blade to provide a flex on the razor blade during a cutting operation. Accordingly, blade grips installed on the non-cutting edges of the razor blade provide locations to securely grasp the razor blade to provide the flex while minimizing grasp slippage and contact with the cutting edges of the razor blade. By way of example, a dermatologist may use the razor blade assembly disclosed herein to take a sample of tissue during a biopsy procedure.

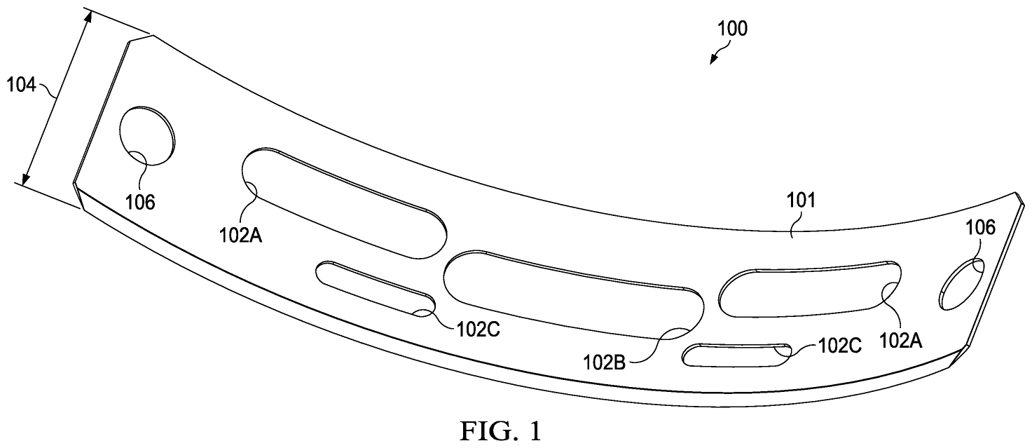

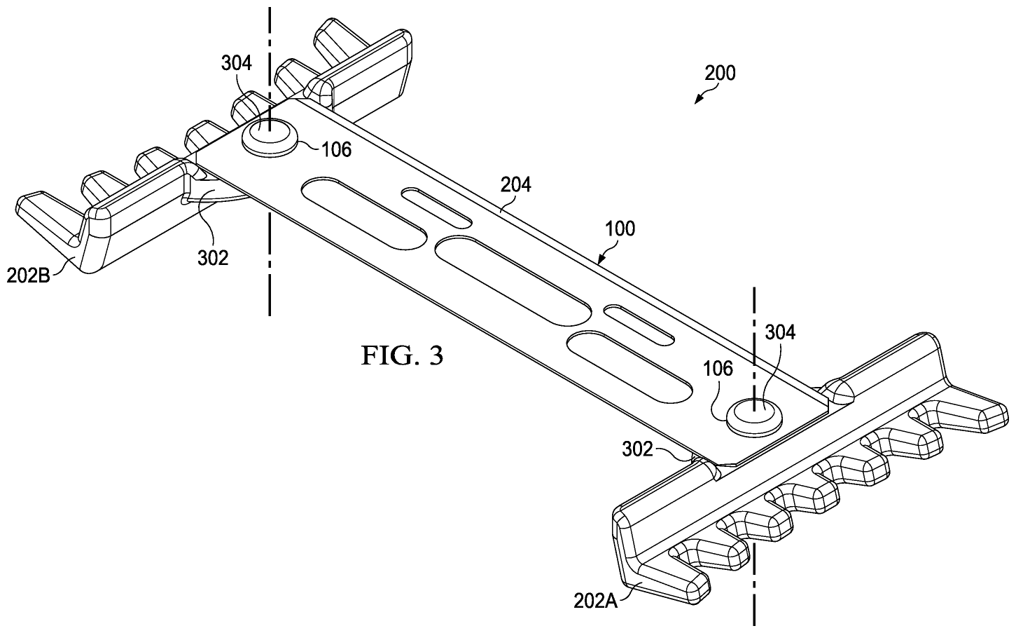

FIG. 1 is a perspective view of a razor blade 100 in a flexed position, in accordance with an embodiment. The razor blade 100 includes a body 101 having openings 102 that may improve the flexibility of the razor blade 100. By way of example, a standard double edged razor blade is 0.004 inches thick. The razor blade 100 may achieve a similar flex as the standard double edged razor blade despite being 0.006 inches thick. The combination of a narrower width 104 than the standard double edged razor blade and the positioning of the openings 102 achieve this desired level of flex. Also included in the razor blade 100 is a pair of mating holes 106 that receive bosses from the grips, as described in detail below with reference to FIG. 3.

The size and positioning of the openings 102 in the body 101 may vary. However, as illustrated in FIG. 1, the openings may include a pair of outboard openings 102A, a central opening 102B, and a pair of inboard openings 102C. The outboard openings 102A are positioned further toward an edge of the body 101 relative to the inboard openings 102C, and the outboard openings 102A are larger than the inboard openings 1020. The central opening 1029 is positioned between the outboard openings 102A and also is positioned centrally on the body 101 of the razor blade 100. The positioning of the openings 102, as shown, results in an acceptable distribution of stresses when the razor blade 100 is placed in a flexed position, but the positioning also provides sufficient strength and flexibility to the razor blade 100.

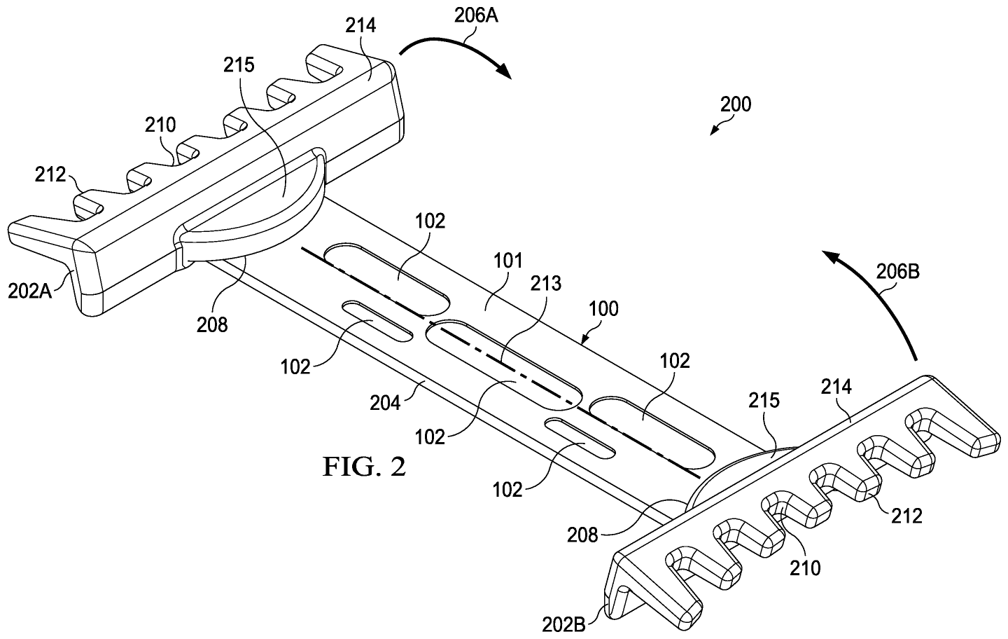

FIG. 2 is a top perspective view of a symmetrical blade grip assembly 200 including the razor blade 100 shown in an unflexed position, in accordance with an embodiment. The symmetrical blade grip assembly 200 also includes blade grips 202A and 202B. The blade grips 202A and 202B couple to the razor blade 100 along opposing edges perpendicular to a cutting edge 204 of the razor blade 100. As discussed above with reference to FIG. 1, the razor blade 100 includes the openings 102, and the shape of the openings 102 may vary. For example, an arrangement and a size of the openings 102 may be adjusted when manufacturing the razor blade 100 to alter rigidity and robustness of the razor blade 100. Altering the rigidity and robustness of the razor blade 100 alters a flex of the razor blade 100 while the razor blade 100 is under a load provided at the blade grips 202A and 202B.

In the illustrated embodiment, a user, such as a dermatologist, is able to grasp the blade grips 202A and 202B with two fingers. The user is then able to flex the razor blade 100 by applying a force in directions 206A and 206B on the blade grips 202A and 202B, respectively, in such a manner that the blade grips 202A and 202B move toward one another. The force applied on the blade grips 202A and 202B results in the razor blade 100 flexing into a shallow ‘u’ shape, or flexed position, as illustrated in FIG. 1.

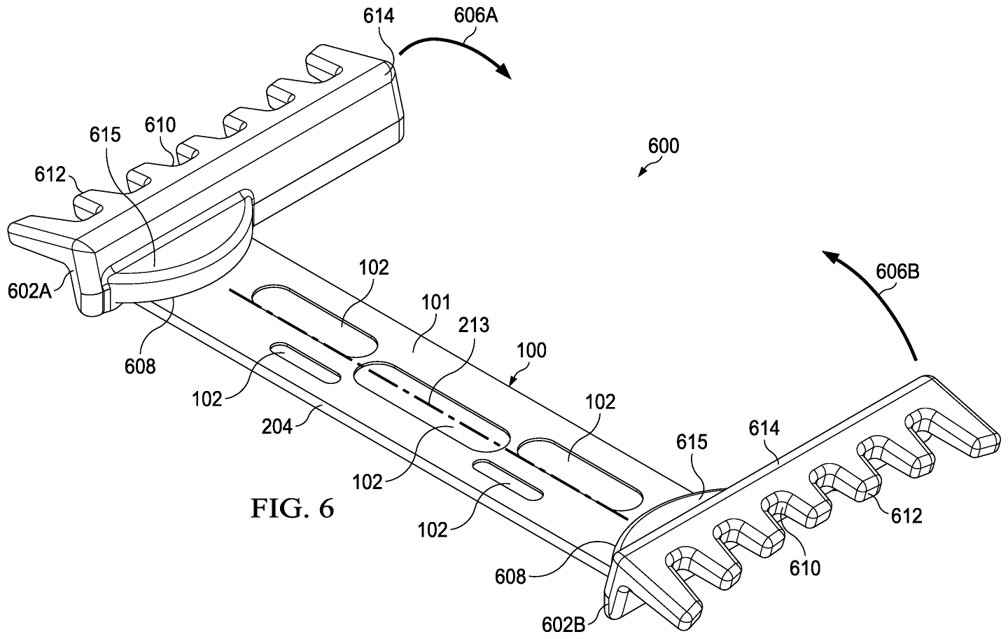

While the razor blade 100 is flexed, the user applies the cutting edge 204 to a surface of an object for cutting. The user may adjust how much pressure is applied on the blade grips 202A and 202B to create a deeper or shallower flex in the razor blade 100. The deeper flex may provide a small and precise cutting surface on the cutting edge 204, while the shallower flex may provide a broader cutting surface on the cutting edge 204. Further, the symmetric positioning of the razor blade 100 between the blade grips 202A and 2029 provides increased balance and feel for the user over an asymmetric positioning of the razor blade 100, as the razor blade 100 is positioned more in line with fingers of the user than the asymmetric positions discussed below with reference to FIG. 6.

The illustrated blade grips 202A and 202B may be formed from the same mold. When the blade grips 202A and 202B are formed from the same mold, the blade grips 202A and 202B are substantially identical. As used herein, the term substantially identical may include minor variations resulting from a manufacturing process. The blade grips 202A and 202B may be made from plastic or any other moldable material. Further, in some embodiments, the blade grips 202A and 202B are made from any moldable material that is capable of being sterilized.

The blade grips 202A and 202B include a blade side 208 and a grip side 210. When coupled to the razor blade 100, the blade side 208 of the blade grip 202 is in contact with the razor blade 100. The grip side 210 of the blade grip 202 includes teeth 212 extending from a body 214 of the blade grip 202. The grip side 210 may be provided in a concave configuration that curves away from the razor blade 100. In such a configuration, outer teeth of the plurality of teeth 212 may be longer than inner teeth 212 thereby forming the concave shape. The concave configuration improves a user's grip and reduces the likelihood of a user's fingers slipping from the grip side 210 of the blade grip 202. In one embodiment, the teeth 212 are integrally formed in the blade grip 202 and break up a surface of the grip side 210 to provide an enhanced gripping surface. For example, the teeth 212 increase friction on the fingers of a user during operation such that slippage of the blade grip 202 is reduced. Additionally, in an embodiment, the grip side 210 both with the teeth 212 and without the teeth 212 may include a rubber coating or an integral rubber portion to provide an enhanced gripping surface. In another embodiment, the entire blade grip 202 is elastomeric to provide the enhanced gripping surface.

The body 214 also includes an arcuate tab 215 that extends from the body 214 in a direction opposite the teeth 212. The arcuate tab 215 provides a connecting point for the razor blade 101. As used herein, the term symmetrical, when describing the symmetrical blade grip assembly 200, refers to the positioning of the blade 100 relative to a width of either blade grip 202A, 2029 (see also FIG. 5). The arcuate tab 215 is centrally located along the width of the body 214, and the razor blade 100 is coupled to the arcuate tab 215 such that a longitudinal line 213 bisecting the razor blade 100 is centrally positioned relative to the width of the blade grip 202A, 202B, Such a positioning of the blade 100 and blade grip 2024 (or 202B) results in a grip assembly that is symmetrical about the longitudinal line 213.

While the tab 215 is described as being arcuate, the tab 215 may be configured in a rectangular, triangular or other shape. In some embodiments, the tab may be an integral part of the body 214 of the blade grip 202A, 202B.

Additionally, while the two blade grips 202A and 202B are depicted in FIG. 2 coupling to the razor blade 100, a single blade grip 202 may also be coupled to the razor blade 100. For example, in an embodiment, the blade grip 202A is coupled to the razor blade 100, and the blade grip 202B is not attached to the razor blade 100. In such an embodiment, a user interacts directly with the razor blade 100 on a side opposite the blade grip 202A. Alternatively, a blade grip 202B with a flat (i.e., not concave) grip side may be installed on the razor blade 100 on the side opposite from the blade grip 202A. In such an embodiment, the teeth 212 and the curvature of the blade grip 202A are only provided on a single side of the razor blade 100.

FIG. 3 is a bottom perspective view of an underside of the symmetrical blade grip assembly 200, in accordance with an embodiment. A coupling side 302 of the blade grips 202A and 202B is a portion of the blade grip 202 that interacts with the razor blade 100 to secure the blade grip 202 to the razor blade 100. The coupling side 302 includes a boss 304 that extends in a direction away from the coupling side 302. In an embodiment, the boss 304 is positioned and shaped to interact with the mating holes 106 of the razor blade 100. The blade may be permanently coupled to the body 214 by deforming (i.e., cold staking) the boss 304 that extends through the mating holes 106 of the razor blade 100.

While the razor blade 100 is described herein as a blade with one cutting edge 204, in some embodiments, the razor blade 100 may be replaced by a blade with dual cutting edges. In such embodiments, the blade grips 202A and 202B may remain mostly unchanged with any differences resulting from a change in a shape and a location of the mating holes 106 on the double edged blade. For example, the bosses 304 may be reshaped and/or moved from the coupling side 302 of the blade grips 202 to align with any mating holes 106 that may be present on the double edged blade.

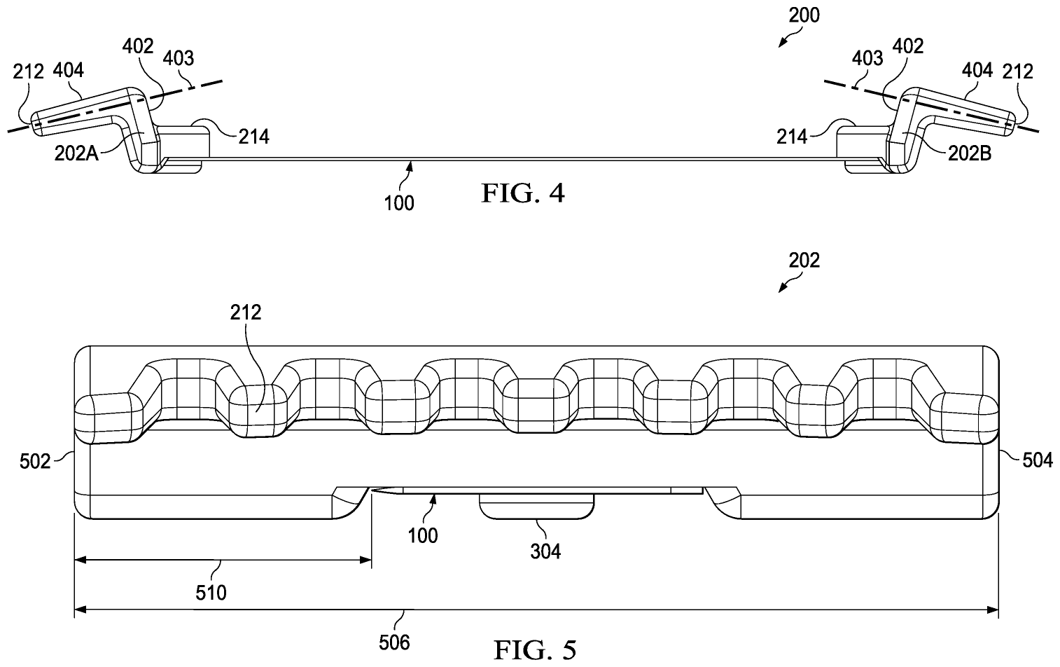

FIG. 4 is a front view of the symmetrical blade grip assembly 200, in accordance with an embodiment. In an embodiment, the blade grips 202A and 202B include steps 402, which extend away from the bodies 214 of the blade grips 202. In one embodiment, a reference line 403 bisects the teeth 212 as illustrated in FIG. 4. The reference line 403 corresponds generally to the middle of the teeth when viewed from the front of the blade grip 202A, 202B. The reference line generally indicates and illustrates an angle at which the teeth are positioned relative to the step 402, the remainder of the body 214, and the planar blade 100 when unflexed. Further, in the illustrated embodiment, the teeth 212 include an angled surface 404. In this embodiment, the angled surface 404 is substantially parallel to the reference line 403, but depending on the configuration and shape of the teeth 212, in some embodiments, the angled surface 404 and reference line 403 may not be parallel. As shown in FIG. 4, the teeth 212 are angled in a direction opposite the direction in which force is applied to the blade grips 202A, 2029 to flex the blade 100 (see also FIG. 2). The angled configuration of the teeth 212 demonstrated by the reference line 403 allow a user to establish a more secure grip while placing the blade 100 in a flexed position since as the blade grips 202A, 202B rotate during flexing of the blade, the teeth 212 are more likely to maintain an orthogonal position relative to the user's fingers and thumb.

Referring still to FIG. 4, the step 402 offsets the teeth 212 from the planar blade 100. More specifically the point at which a user's fingers or thumbs contact the teeth will be approximately defined by where the reference line 403 meets an outer edge of the teeth 212. Since this point (and the teeth 212 generally) are separated from the planar blade in a direction perpendicular to the blade, the offset provides additional leverage for the user when placing the blade 100 in the flexed position.

Further, the step 402 also offsets the teeth 212 from the point at which the planar blade 100 connects to the blade grip 202A, 202B in a direction parallel to the planar blade. Again, this offset improves the leverage of the user as the razor blade 100 is flexed. That is, the offset improves the leverage during flexing relative to a blade grip with the teeth 212 positioned in line with the tab 215 (e.g., without the step 402). Accordingly, user fatigue may be avoided during use of the blade grip assembly 200 as less force is exerted on the blade grip 202 to achieve a desired flex in the razor blade 100.

FIG. 5 is a side view of the blade grip 202 of the symmetrical blade grip assembly 200, in accordance with an embodiment. As discussed above, because the blade grips 202A and 202B are substantially identical, the illustrated blade grip 202 may be either the blade grip 202A or the blade grip 202B. As illustrated, the blade grip 202 includes the teeth 212 positioned on a different horizontal plane from the razor blade 100 when the razor blade 100 is in an unflexed position. The razor blade 100 is coupled to the blade grip 202 via the boss 304. Because the symmetrical blade grip assembly 200 is symmetrical, the razor blade 100 and the boss 304 are both centered between edges 502 and 504 of the blade grip 202.

Also shown in FIG. 5 is the positioning of the cutting edge 204 of the blade 100 relative to a width 506 of the blade grip 202. The distance 510 of the cutting edge 204 of the blade 100 from a nearest end of the blade grip 202 is such that cutting edge 204 is nearer to a center of the blade grip than the edge of the blade grip in this particular symmetric embodiment. In other embodiments the cutting edge 204 may be positioned closer to the nearest edge of the blade grip, thereby decreasing the distance 510. In some embodiments it is desirable to minimize the distance 510 to provide better visibility of the item being cut by the user. The distance 510 may be decreased even with the symmetrical embodiments of the blade grip 202 by increasing a width of the blade or decreasing the width 506 of the blade grip 202.

FIG. 6 is a top perspective view of an asymmetrical blade grip assembly 600 including the razor blade 100, in accordance with an embodiment. The asymmetrical blade grip assembly 200 also includes blade grips 602A and 602B. The blade grips 602A and 602B couple to the razor blade 100 along surfaces perpendicular to the cutting edge 204 of the razor blade 100. As discussed above with reference to FIG. 1, the razor blade 100 includes openings 102, and the shape of the openings 102 may vary. For example, an arrangement and a size of the openings 102 may be adjusted when manufacturing the razor blade 100 to alter rigidity and robustness of the razor blade 100. Altering the rigidity and robustness of the razor blade 100 alters a flex of the razor blade 100 while the razor blade 100 is under a load provided at the blade grips 602A and 602B.

In the illustrated embodiment, a user, such as a dermatologist, is able to grasp the blade grips 602A and 602B with two fingers. The user is then able to flex the razor blade 100 by applying a force in directions 606A and 60613 on the blade grips 602A and 602B, respectively, in such a manner that the blade grips 602A and 602B move toward one another. The force applied on the blade grips 602A and 602B results in the razor blade 100 flexing into a shallow shape, as illustrated in FIG. 1.

While the razor blade 100 is flexed, the user applies the cutting edge 204 to a surface of an object for cutting. For example, the user may adjust how much pressure is applied on the blade grips 602A and 602B to create a deeper or shallower flex in the razor blade 100. The deeper flex may provide a small and precise cutting surface on the cutting edge 204, while the shallower flex may provide a broader cutting surface on the cutting edge 204. Additionally, the asymmetric positioning of the razor blade 100 between the blade grips 602A and 602B provides enhanced visibility of the cutting edge 204 during a procedure as the razor blade 100 is positioned in front of the fingers flexing the razor blade 100 in relation to a cutting direction of the razor blade 100.

The blade grips 602A and 602B may be made from plastic or any other moldable material. Further, in some embodiments, the blade grips 602A and 602B are made from any moldable material that is capable of being sterilized.

The blade grips 602A and 602B include a blade side 608 and a grip side 610. When coupled to the razor blade 100, the blade side 608 of the blade grip 602 is in contact with the razor blade 100. The grip side 610 of the blade grip 602 includes teeth 612 extending from a body 614 of the blade grip 602. The grip side 610 may curve away from the razor blade 100, which reduces a likelihood of a user's fingers slipping from the grip side 610 of the blade grip 602. Further, the teeth 612 are integrally formed in the blade grip 602 and break up a surface of the grip side 610 to provide an enhanced gripping surface. For example, the teeth 612 increase friction on the fingers of a user during operation such that slippage of the blade grip 602 is reduced. Additionally, in an embodiment, the grip side 610 both with the teeth 612 and without the teeth 612 may include a rubber coating or an integral rubber portion to provide an enhanced gripping surface. In another embodiment, the entire blade grip 602 is elastomeric to provide the enhanced gripping surface.

The body 614 also includes an arcuate tab 615 that extends from the body 614 in a direction opposite the teeth 612. The arcuate tab 615 provides a connecting point for the razor blade 100. As used herein, the term asymmetrical, the term symmetrical, when describing the asymmetrical blade grip assembly 600, refers to the positioning of the blade 100 relative to a width of either blade grip 602A, 602B (see also FIG. 9). The arcuate tab 615 is offset from a central portion of the body 614 along the width of the body 214, and the razor blade 100 is coupled to the arcuate tab 615 such that a longitudinal line 613 bisecting the razor blade 100 is offset from the central point of the body 614 along the width of the body 614. Such a positioning of the blade 100 and blade grip 602A (or 602B) results in a grip assembly that is asymmetrical about the longitudinal line 613.

While the tab 615 is described as being arcuate, the tab 615 may be configured in a rectangular, triangular or other shape. In some embodiments, the tab may be an integral part of the body 614 of the blade grip 602A, 602B.

Additionally, while the two blade grips 602A and 602B are depicted in FIG. 6 coupling to the razor blade 100, a single blade grip 602 may also be coupled to the razor blade 100. For example, in an embodiment, the blade grip 602A is coupled to the razor blade 100, and the blade grip 602B is not attached to the razor blade 100. In such an embodiment, a user interacts directly with the razor blade 100 on a side opposite the blade grip 602A. Alternatively, a blade grip 602B with a flat (i.e., not concave) grip side may be installed on the razor blade 100 on the side opposite from the blade grip 602A. In such an embodiment, the teeth 612 and the curvature of the blade grip 602A are only provided on a single side of the razor blade 100.

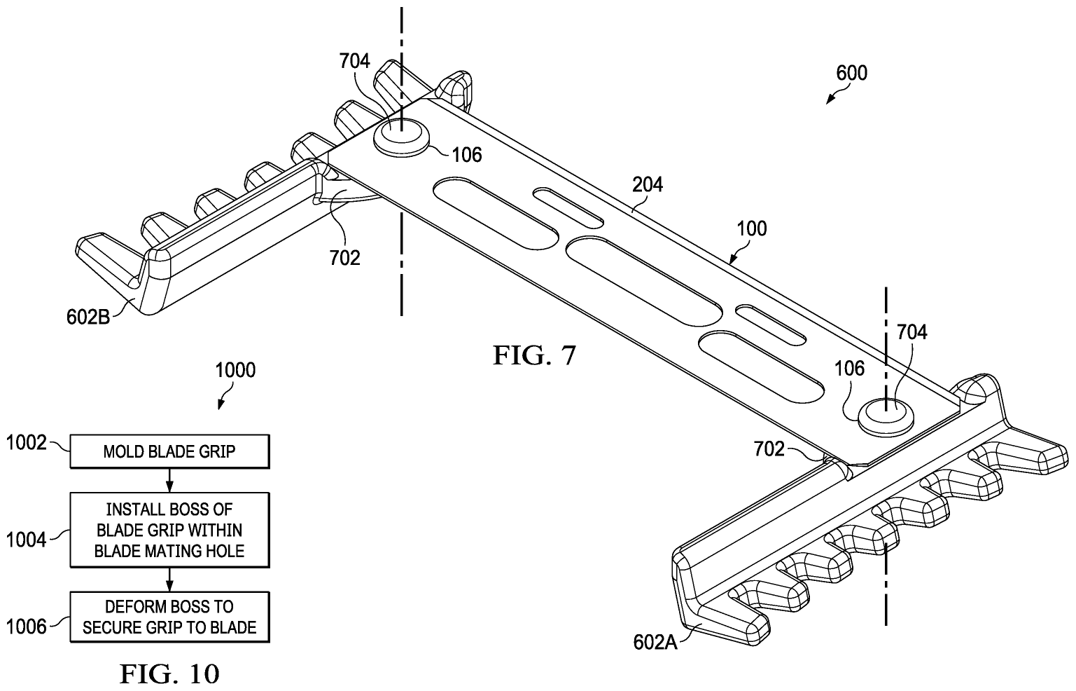

FIG. 7 is a bottom perspective view of an underside of the asymmetrical blade grip assembly 600, in accordance with an embodiment. A coupling side 702 of the blade grips 602A and 602B is a portion of the blade grip 602 that interacts with the razor blade 100 to secure the blade grip 602 to the razor blade 100. The coupling side 702 includes a boss 704 that extends in a direction away from the coupling side 702. In an embodiment, the boss 704 is positioned and shaped to interact with the mating holes 106 of the razor blade 100.

While the razor blade 100 is described herein as a blade with a single cutting edge 204, in some embodiments, the razor blade 100 may be replaced with a blade that includes multiple cutting edges. In such embodiments, the blade grips 602A and 602B may remain mostly unchanged with any differences resulting from a change in a shape and a location of the mating holes 106 on the double edged blade. For example, the bosses 704 may be reshaped and/or moved from the coupling side 702 of the blade grips 602 to align with any mating holes 106 that may be present on the double edged blade.

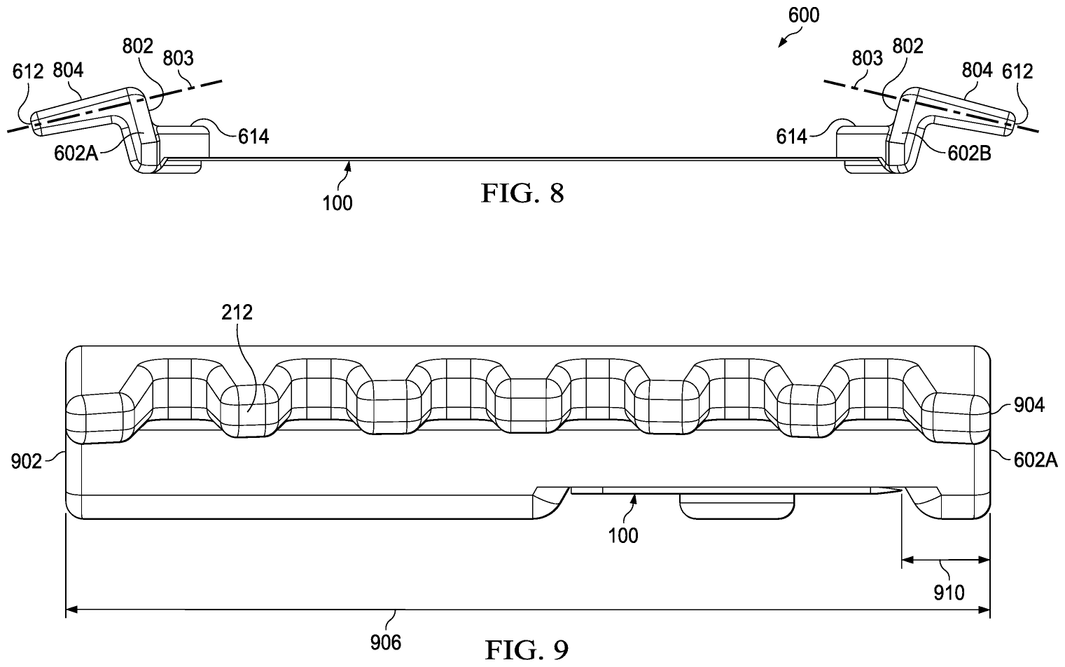

FIG. 8 is a front view of the asymmetrical blade grip assembly 600, in accordance with an embodiment. In an embodiment, the blade grips 602A and 602B include steps 802, which extend away from the bodies 614 of the blade grips 602. In one embodiment, a reference line 803 bisects the teeth 612 as illustrated in FIG. 8. The reference line 803 corresponds generally to the middle of the teeth when viewed from the front of the blade grip 602A, 602B. The reference line generally indicates and illustrates an angle at which the teeth are positioned relative to the step 802, the remainder of the body 614, and the planar blade 100 when unflexed. Further, in the illustrated embodiment, the teeth 612 include an angled surface 804. In this embodiment, the angled surface 804 is substantially parallel to the reference line 803, but depending on the configuration and shape of the teeth 612, in some embodiments, the angled surface 804 and reference line 803 may not be parallel. As shown in FIG. 8, the teeth 612 are angled in a direction opposite the direction in which force is applied to the blade grips 602A, 602B to flex the blade 100 (see also FIG. 6). The angled configuration of the teeth 612 demonstrated by the reference line 803 allow a user to establish a more secure grip while placing the blade 100 in a flexed position since as the blade grips 602A, 602B rotate during flexing of the blade, the teeth 612 are more likely to maintain an orthogonal position relative to the user's fingers and thumb.

Referring still to FIG. 8, the step 802 offsets the teeth 612 from the planar blade 100. More specifically the point at which a user's fingers or thumbs contact the teeth will be approximately defined by where the reference line 803 meets an outer edge of the teeth 612. Since this point (and the teeth 612 generally) are separated from the planar blade in a direction perpendicular to the blade, the offset provides additional leverage for the user when placing the blade 100 in the flexed position.

Further, the step 802 also offsets the teeth 612 from the point at which the planar blade 100 connects to the blade grip 602A, 602B in a direction parallel to the planar blade. Again, this offset improves the leverage of the user as the razor blade 100 is flexed. That is, the offset improves the leverage during flexing relative to a blade grip with the teeth 612 positioned in line with the tab 615 (e.g., without the step 802). Accordingly, user fatigue may be avoided during use of the blade grip assembly 600 as less force is exerted on the blade grip 602 to achieve a desired flex in the razor blade 100.

FIG. 9 is a side view of the blade grip 602A of the asymmetrical blade grip assembly 600, in accordance with an embodiment. Because the blade grips 602A and 602B are not substantially identical, a side view of the blade grip 602B would be a mirror image of the blade grip 602A illustrated in FIG. 9. As illustrated, the blade grip 602A includes the teeth 212 positioned on a different horizontal plane from the razor blade 100. The razor blade 100 is coupled to the blade grip 602A via the boss 704. Because the asymmetrical blade grip assembly 600 is asymmetrical, the razor blade 100 and the boss 704 are both positioned closer to edge 904 than edge 902 of the blade grip 602A. Additionally, the razor blade 100 and the boss 704 of the blade grip 602B is positioned closer to the edge 902 than the edge 904 when viewed from a similar angle as the blade grip 602A in FIG. 9.

Also shown in FIG. 9 is the positioning of the cutting edge 204 of the blade 100 relative to a width 906 of the blade grip 602. The distance 910 of the cutting edge 204 of the blade 100 from a nearest end of the blade grip 602 is such that cutting edge 204 is near to the edge of the blade grip 602. In one embodiment, the distance 910 may be approximately 0.010-4100 inches. In such an embodiment the width 906 may in some embodiments be approximately 0.875 inches, which results in the cutting edge being positioned a distance from the edge that is approximately 1% to 12% the width 906 of the blade grip 602. In other embodiments, the distance 910 may be approximately 0.0-0.5 inches, while in other embodiments it may be desired to have the cutting edge 204 positioned past the edge of the blade grip 602. However, it is desired in some embodiments to have a portion of the blade grip covering or preventing exposure to a corner of the blade 100; therefore, in these embodiments, the cutting edge 204 may maintain a small distance between the cutting edge 204 and the edge of the blade grip 602. The asymmetric configuration of the blade grip 602 allows for reduction in the distance 910 to provide better visibility of the item being cut by the user.

FIG. 10 is a flow chart of a method 1000 of manufacturing the symmetrical blade grip assembly 200 or the asymmetrical blade grip assembly 600, in accordance with an embodiment. Initially, the blade grips 202 or 602 are molded. Any molding process capable of producing the blade grips 202 or 602 is contemplated as within the scope of this disclosure. For example, the blade grips 202 or 602 may be formed using injection molding or compression molding techniques.

After the blade grips 202 or 602 are molded, the boss 304 is installed within the mating hole 106 of the razor blade 100 at block 1004. The boss 304 has a close fit with the mating hole 106. For example, contact between the boss 304 of the blade grip 202 or 602 and the mating hole 106 of the razor blade 100 may occur as the blade grips 202A/602A and 2023/602B are installed on the razor blade 100.

The contact between the razor blade 100 and the blade grips 202A/602A and 202B/602B may provide partial securement of the blade grips 202A/602A and 202B/602B on the razor blade 100. As used herein, partial securement may refer to the blade grips 202A/602A and 202B/602B maintaining contact with the razor blade 100 when no external forces are applied to the blade grips 202A/602A and 202B/602B or the razor blade 100, but, upon applying external forces, the blade grips 202A/602A and 202B/602B and the razor blade 100 may be separable without deformation of either the blade grips 202A/602A and 202B/602B or the razor blade 100. The partial securement of the blade grips 202A/602A and 202B/602B and the razor blade 100 may ensure that sufficient amounts of material from the bosses 304 will overlap the razor blade 100 upon plastically deforming the bosses 304 to permanently secure the blade grips 202A/602A and 202B/602B to the razor blade 100.

At block 1006, the bosses 304 are plastically deformed to permanently secure the blade grips 202A/602A and 202B/602B to the razor blade 100. As used herein, permanently securing the blade grips 202A/602A and 202B/602B to the razor blade 100 may refer to securing the blade grips 202A/602A and 2023/602B to the razor blade 100 in an irreversible manner. Further, in an embodiment, the term permanent securement may be defined as securement that sufficiently resists separation when the device is operating under an amount of force considered within a normal operating parameter of the device. That is, permanent securement, in some embodiments, means that the blade grips 202A/602A and 202B/602B will not separate from the razor blade 100 when the blade grip assembly 200/600 is used during a biopsy procedure.

To deform the bosses 304, a cold staking procedure may be performed on the bosses 304 when properly aligned with the razor blade 100. The cold staking procedure involves applying pressure on the bosses 304 at room temperature to compress the bosses 304. When the bosses 304 are compressed, the plastic from the bosses 304 extends over edges of the corresponding mating holes 106 of the razor blade 100. Extension of the plastic from the bosses 304 over the edges of the razor blade 100 permanently secures the blade grip 202/602 to the razor blade 100. Further, the bosses 304 may also be deformed with a heat staking process (i.e., applying heat and pressure to the bosses 304) or through an ultrasonic staking process (i.e., controlled melting of the bosses 304 using ultrasonic vibrations), or any other plastic deformation process. Furthermore, in place of deforming the bosses 304, the blade grip 202/602 may be glued to the razor blade 100 to permanently secure the blade grip 202/602 to the razor blade 100.

In another embodiment, each of the blade grips 202/602 may include two separate pieces that are coupled together on either end of the razor blade 100. For example, the blade grips 202/602 may include one piece with the boss 304 and another piece that is positioned on an opposite side of the razor blade 100 with recesses shaped to receive the boss 304. In such an embodiment, the two pieces of the blade grips 202/602 may be attached to each other and to the razor blade 100 using ultrasonic welding, staking, or gluing.

As discussed above with reference to FIG. 1, the openings 102 provide the razor blade 100 with similar flex to a standard double edged razor blade wider similar loads. By way of example, a standard double edged razor blade may be approximately 0.004 inches thick. The razor blade 100 may achieve a similar flex as the standard double edged razor blade despite being thicker, such as a blade that is approximately 0.006 inches thick. The combination of a narrower width 104 than a standard double edged razor blade and the positioning of the openings 102 achieve this desired level of flex.

While this specification provides specific details related to certain components of the blade grip assemblies 200 and 600, it may be appreciated that the list of components is illustrative only and is not intended to be exhaustive or limited to the forms disclosed. Other components of the blade grip assemblies 200 and 600 will be apparent to those of ordinary skill in the art without departing from the scope and spirit of the disclosure. Further, the scope of the claims is intended to broadly cover the disclosed components and any such components that are apparent to those of ordinary skill in the art.

The above disclosed embodiments have been presented for purposes of illustration and to enable one of ordinary skill in the art to practice the disclosed embodiments, but is not intended to be exhaustive or limited to the forms disclosed. Many insubstantial modifications and variations will be apparent to those of ordinary skill in the art without departing from the scope and spirit of the disclosure. For instance, although the flow diagram depicts serial processes, some of the steps/blocks may be performed in parallel or out of sequence, or combined into a single step/block. The scope of the claims is intended to broadly cover the disclosed embodiments and any such modification.

It should be apparent from the foregoing disclosure of illustrative embodiments that significant advantages have been provided. The illustrative embodiments are not limited solely to the descriptions and illustrations included herein and are instead capable of various changes and modifications without departing from the spirit of the disclosure.

What is claimed is:

1. A blade grip assembly comprising:
a blade having at least one cutting edge; and
at least one blade grip coupled to the blade, the blade grip having a body and a plurality of teeth extending from the body;
wherein the plurality of teeth is on a plane different from a plane of the blade when the blade is in an unflexed position.

2. The blade grip assembly according to claim 1, wherein the plane of the plurality of teeth is parallel to the plane of the blade.

3. The blade grip assembly according to claim 1, wherein the plane of the plurality of teeth is angled relative to the plane of the blade.

4. The blade grip assembly according to claim 3, wherein the teeth are angled away from a desired direction of flexure of the blade.

5. The blade grip assembly according to claim 1 further comprising a tab centrally located on the body of each blade grip such that the blade grips are symmetrical about a longitudinal axis of the blade.

6. The blade grip assembly according to claim 5, wherein the tab is arcuate.

7. The blade grip assembly according to claim 1 further comprising a tab positioned on the body of each blade grip such that the blade grip is asymmetrical about a longitudinal axis of the blade.

8. The blade grip assembly according to claim 7, wherein the tab is arcuate.

9. The blade grip assembly according to claim 1, wherein the plurality of teeth of each blade grip is arranged in a concave configuration.

10. A blade grip assembly comprising:
a blade having at least one cutting edge; and
at least one blade grip coupled to the blade, the blade grip having a body and a plurality of teeth extending from the body;
wherein the plurality of teeth is on a plane angled relative to a plane of the blade when the blade is in an unflexed position.

11. The blade grip assembly according to claim 10, wherein the teeth are angled away from a direction of flexure of the blade.

12. The blade grip assembly according to claim 10 further comprising a tab centrally located on the body of each blade grip such that the blade grips are symmetrical about a longitudinal axis of the blade.

13. The blade grip assembly according to claim 12, wherein the tab is arcuate.

14. The blade grip assembly according to claim 10 further comprising a tab positioned on the body of each blade grip such that the blade grip is asymmetrical about a longitudinal axis of the blade.

15. The blade grip assembly according to claim 14, wherein the tab is arcuate.

16. The blade grip assembly according to claim 1, wherein the plurality of teeth of each blade grip is arranged in a concave configuration.

17. A blade grip assembly comprising:
a blade having at least one cutting edge; and
at least one blade grip coupled to the blade, the blade grip having a body and a plurality of teeth extending from the body;
wherein the cutting edge of the blade is positioned a distance from an edge of the blade grip that is approximately 1-12% a width of the blade grip.

18. The blade grip according to claim 17, wherein the plurality of teeth is on a plane different from a plane of the blade.

19. The blade grip of claim 17, wherein the distance from the edge is 0.010-0.100 inches.

20. The blade grip of claim 17, wherein the blade is positioned relative to the body such that the blade grip is asymmetrical about a longitudinal axis of the blade.
